(19) United States
(12) Patent Application Publication
(10) Pub. No.: US 2023/0110977 A1
(43) Pub. Date: Apr. 13, 2023

(54) METHODS OF IMPROVING BONE HEALTH

(71) Applicant: ABBOTT LABORATORIES, Abbott Park, IL (US)

(72) Inventors: José María LÓPEZ PEDROSA, Granada (ES); Manuel MANZANO MARTÍN, Granada (ES); Ricardo RUEDA CABRERA, Granada (ES)

(21) Appl. No.: 17/802,716

(22) PCT Filed: Feb. 26, 2021
(86) PCT No.: PCT/US2021/019908

§ 371 Date: Aug. 26, 2022

(30) Foreign Application Priority Data
Feb. 27, 2020 (EP) 20382139.2

(51) Int. Cl. A61K 31/702; A61K 31/7004; A61K 31/7016; A61K 31/718; A61P 19/08; A23L 33/125; A23L 33/24

(52) U.S. Cl. CPC A61K 31/702; A61K 31/7004; A61K 31/7016; A61K 31/718; A61P 19/08; A23L 33/125; A23L 33/24; A23V 2002/00

(57) ABSTRACT

A method of promoting bone health in a moderately malnourished individual is provided. The method includes treating the moderately malnourished individual with a nutritional composition that contains a carbohydrate blend. The carbohydrate blend includes a source of at least one carbohydrate that provides rapidly available glucose, a source of at least one carbohydrate that provides slowly available glucose, and a source of at least one non-digestible carbohydrate or resistant starch.

### CROSS-REFERENCE TO RELATED APPLICATIONS

This application claims priority to and the benefit of European Patent Application No. 20382139.2, filed on Feb. 27, 2020, the entire content of which is incorporated herein by reference.

### FIELD

The present disclosure relates to methods of promoting bone health in individuals. More particularly, the present disclosure relates to promoting bone health in a moderately malnourished individual by treating the moderately malnourished individual with a nutritional composition comprising a carbohydrate blend.

### BACKGROUND

Growth stunting constitutes the most common effect of malnutrition. However, when the primary cause of malnutrition is resolved, catch-up growth usually occurs. Catch-up growth places affected individuals at a higher risk of developing osteoporosis and obesity as compared to those who do not experience catch-growth in their lifetime. Catch-up growth has existed as a suggested benefit to the adipogenic differentiation of Mesenchymal Stem Cells (MSCs), a common cell progenitor of adipocytes and osteoblasts, and thus, suppresses the osteoblastic differentiation of MSCs, which may be responsible for causing the increased risk of osteoporosis and obesity during catch-up growth after food restriction.

Adequate nutrition, in amount and composition, is essential for skeletal health in all individuals, and the importance of meeting daily dietary requirements is amplified in those stunted individuals during the catch-up growth period. Collective intake of vitamins, minerals, trace elements, and macronutrients must be considered in order to develop effective nutritional strategies used to preserve and improve bone health in such individuals. Moreover, improving the structural integrity and strength of such individuals' bones can positively influence bone health.

Bone growth and development, as well as overall health, are positively affected by implementing dietary choices which favor high quality protein of animal or plant origin, polyunsaturated fatty acids, fruits and vegetables high in potassium and fiber, and daily products or other nutritional supplements fortified with calcium and vitamins (A, C, D, E, and K).

Determining how to properly optimize peak bone mass and bone strength early in life, as well as stabilize it during young adulthood, to prevent osteoporosis and fractures later in life positively contributes to promoting and ensuring proper bone health. A 10% increase in peak bone mineral mass of children could delay the development of osteoporosis by thirteen years, as well as reduce the risk of fracture in such children by 50%. Moreover, the foundation for achieving lifelong skeletal health is established during childhood (which for purposes of this document, includes adolescence). Because approximately 90% of adult bone mass is gained in the first two decades of life, in addition to bone size and strength reaching a maximum by early adulthood, the critical period for bone accretion that determines optimal peak bone mass and health occurs through childhood (including through late adolescence).

Both body and skeletal growth are significantly affected in stunted individuals. Stunting results from a complex interaction among undernutrition, poor gut health, infectious disease, and compromised immune function. An inadequate dietary supply is detrimental to the acquisition of bone mass during growth, leading to linear childhood growth retardation and its respective conservation during adulthood. Consequently, malnutrition, which occurs during an individual's period of growth and development, can increase the individual's risk of osteoporosis, rickets, and osteomalacia. In growing individuals, osteoporosis refers to a low bone mass for achieved size with high porosity. Osteoporosis is associated with an increased risk of fragility fracture in children and adults, i.e. a fracture caused with minimal trauma, such as a fall from a standing position. Osteomalacia is caused by a decelerated mineralization on the trabecular and cortical surfaces of all bones. Meanwhile, rickets occurs when the long bones of an individual's growth plates fail to calcify and produce characteristic bone deformities which occur before the growth plates are properly fused.

In stunted children, these periods of skeletal growth retardation are usually followed by compensatory catch-up growth. However, in many cases, depending on the child's age and the extent of the growth deficit, catch-up growth is insufficient to reach the optimal peak bone mass and bone strength, yielding a permanent deficit on bone health.

However, the number of children from ages 5-19 who are moderately or severely underweight remains larger than those who are obese, demonstrating the need for nutritional intervention guidance to enhance food security. Yet, the transition from underweight to overweight is often rapid and can affect a healthy transition. Although the first line of treatment is dietary counseling, a trial of oral supplementation to improve the dietary intake of children who are unable to meet their nutritional requirements with normal foods alone should follow such counseling. Energy- and protein-enriched (1.5 kcal/ml; PE % between 8-12%) formulae are appropriate for underweight children to facilitate and improve weight gain and linear growth. Such formulae provide better energy/protein ratio, a more concentrated micronutrient profile, and a lower osmolality. In addition, they are ready to use—further minimizing the risk of bacterial contamination. However, in an unhealthy nutritional transition, an increase in nutrient-poor, energy-dense foods can lead to stunted growth, along with weight gain in children which results in higher BMI and/or accelerated fat recovery that predisposes individuals with catch-up growth to insulin resistance and risk for later metabolic syndrome.

### SUMMARY

Accordingly, there is an unmet need for a method of promoting bone health which promotes bone health by increasing bone density and/or content, increasing bone length, improving bone microarchitecture, or a combination thereof. The present invention provides a nutritional composition for use in, and methods associated with, treating moderately malnourished individuals by improving bone health in young children, during a period of catch-up growth or weight recovery, following a period of growth restriction or weight loss, leading to improved bone status. Growth improvement is promoted by enhancing both bone and mineral accretion and quality in both appendicular and axial bones, resulting in healthy linear growth.

The present invention relates to promoting bone health using a carbohydrate system (comprising a ratio of rapidly-digested and slowly-digested carbohydrates and mono and disaccharides and non-digestible oligosaccharides or resistant starch) for the preparation of a nutritional composition for administration to a young child during a period of catch-up growth or weight recovery, following a period of growth restriction or weight lost, leading to an improved bone status (both quantity and quality). Optimizing peak bone mass and bone strength in stunted children will play a significant role in preventing osteoporosis and fractures later in life.

Disclosed herein are nutritional compositions comprising a carbohydrate blend used to treat a moderately malnourished individual by improving bone health during a period of catch-up growth or weight recovery.

In accordance with the present disclosure, a method of treating moderately malnourished individuals by improving bone health during a period of catch-up growth is provided. The method includes treating the moderately malnourished individual by administering a nutritional composition comprising a carbohydrate blend of: (i) a source of at least one carbohydrate that provides rapidly available glucose; (ii) a source of at least one carbohydrate that provides slowly available glucose; and (iii) a source of at least one non-digestible carbohydrate or resistant starch.

The present disclosure also provides a method of promoting bone health in a moderately malnourished individual, the method comprising administering a carbohydrate blend of: (i) a source of at least one carbohydrate that provides rapidly available glucose; (ii) a source of at least one carbohydrate that provides slowly available glucose; and (iii) a source of at least one non-digestible carbohydrate or resistant starch. The method of promoting bone health of a moderately malnourished individual includes treating malnourishment by promoting healthy catch-up growth, particularly with respect to bone health. Further, by treating malnourishment, the moderately malnourished individual's bone health is improved.

The present disclosure also provides use of a nutritional composition for the manufacture of a medicament for treating malnourishment in a moderately malnourished individual by improving bone health in that individual, the nutritional composition comprising: (i) a source of at least one carbohydrate that provides rapidly available glucose; (ii) a source of at least one carbohydrate that provides slowly available glucose; and (iii) a source of at least one non-digestible carbohydrate or resistant starch. The method of promoting bone health moderately malnourished individual includes treating malnourishment by administering the nutritional composition to the individual thereby promoting healthy catch-up growth, particularly with respect to bone health.

In accordance with the present disclosure, the method of promoting bone health in a moderately malnourished individual is provided. The method includes administering to the moderately malnourished individual a nutritional composition comprising a protein, a fat, and a carbohydrate blend comprising: (i) a source of at least one carbohydrate that provides rapidly available glucose; (ii) a source of at least one carbohydrate that provides slowly available glucose; and (iii) a source of at least one non-digestible carbohydrate or resistant starch.

The present disclosure also provides a nutritional composition for use in treating a moderately malnourished individual by improving bone health wherein the nutritional composition comprises a protein, a fat, and a carbohydrate blend comprising: (i) a source of at least one carbohydrate that provides rapidly available glucose; (ii) a source of at least one carbohydrate that provides slowly available glucose; and (iii) a source of at least one non-digestible carbohydrate or resistant starch. Promoting bone health includes treating malnourishment by administering the nutritional composition to the individual thereby promoting healthy catch-up growth, particularly with respect to bone health.

The present disclosure also provides the use of a nutritional composition for the manufacture of a medicament for treating a moderately malnourished individual by improving bone health, the nutritional composition comprising a protein, a fat, and a carbohydrate blend comprising: (i) a source of at least one carbohydrate that provides rapidly available glucose; (ii) a source of at least one carbohydrate that provides slowly available glucose; and (iii) a source of at least one non-digestible carbohydrate or resistant starch. The malnourishment is treated by promoting healthy catch-up growth particularly with respect to bone health.

### BRIEF DESCRIPTION OF THE DRAWINGS

FIG. 1 depicts the percentage of changes of the tibia length in rapidly digestible carbohydrate (RDC) and slowly digestible carbohydrate (SDC) groups after the re-feeding period found in Example 1.

FIG. 2 provides various graphs depicting the percentage change in tibia bone parameters in RDC and SDC groups with respect to the restricted (RR) group in bone quantity and quality parameters after the re-feeding period in Example 1.

FIG. 3 provides various graphs depicting the percentage of changes in lumbar vertebra bone parameters in RDC and SDC groups with respect to the RR in bone quantity and quality parameters after the re-feeding period at the end of the study of Example 1.

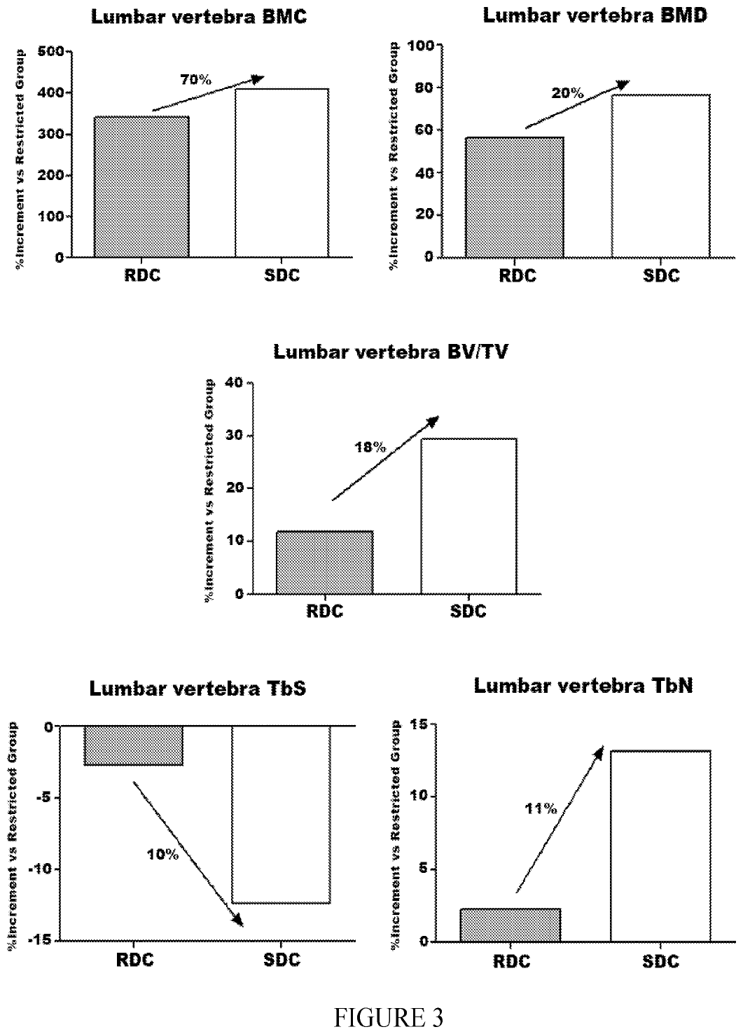

### DETAILED DESCRIPTION

Disclosed herein are nutritional compositions used to promote bone health in a moderately malnourished individual.

The term “individual” as used herein, refers generally to a preterm infant, infant, toddler, or child.

The term “infant” as used herein, refers generally to individuals up to 36 months of age, actual or corrected.

The term “preterm infant” as used herein, refers to infants born at less than 37 weeks gestation, infants that have a birth weight of less than 2,500 grams, or both.

The term “nutritional composition” as used herein, unless otherwise specified, refers to synthetic formulas including nutritional liquids, nutritional powders, nutritional solids, nutritional semi-solids, nutritional semi-liquids, nutritional supplements, and any other nutritional food product as known in the art. The nutritional powders may be reconstituted to form a nutritional liquid, all of which comprise one or more of fat, protein, and carbohydrate and are suitable for oral consumption by a human. The term “nutritional composition” does not include human breast milk and does not refer to supplemented milk.

The term “nutritional liquid” as used herein, unless otherwise specified, refers to nutritional compositions in ready-to-drink liquid form, concentrated form, and nutritional liquids made by reconstituting the nutritional powders described herein prior to use.

The term “nutritional powder” as used herein, unless otherwise specified, refers to nutritional compositions in flowable or scoopable form that can be reconstituted with water or another aqueous liquid prior to consumption and includes both spray dried and dry mixed/dry blended powders.

The term “shelf stable” as used herein, unless otherwise specified, refers to a nutritional composition that remains commercially stable after being packaged and then stored at 18-24° C. for at least 3 months, including from about 3 months to about 24 months, and also including from about 3 months to about 18 months.

The term “catch-up growth” as used herein, unless otherwise specified, refers to a remedial level or rate of growth or development to achieve a basal level of growth following a transient period of growth restriction or inhibition. In reference to stunted children, catch up-growth is defined as the height velocity above the normal statistical limits for age and/or maturity during a defined period of time, following a transient period of growth inhibition which returns them to their original skeletal growth trajectory.

The term “healthy catch-up growth” as used herein, unless otherwise specified, refers to catch-up growth in which the phenomenon of catch-up fat is avoided or reduced.

The term “catch-up fat” as used herein, unless otherwise specified, refers to a disproportionately higher rate in the recovery of body fat than lean tissue during a period of catch-up growth.

The terms “moderate malnutrition” and “moderately malnourished” as used herein, unless otherwise specified, refer to a weight-for-age between −3 and −2 z-scores below the median of the World Health Organization (WHO) child growth standards. Moderate malnutrition can be due to a low weight-for-height (wasting) or a low height-for-age (stunting) or to a combination of both. Similarly, moderate wasting and stunting are defined as a weight-for-height and height-for-age, respectively, between −3 and −2 z-scores. For example, stunting is characterized by a low height-for-age, such as a height-for-age z-score of less than −2 based on the WHO child growth standards median. Similarly, wasting is characterized by a low weight-for-height, such as a weight-for-height z-score of less than −2 based on the WHO child growth standards median.

The term “bone health” refers to any of bone density and/or content, bone length, bone microarchitecture, and combinations thereof.

Promoting bone health includes improving bone health and may be achieved by increasing bone density and/or content, increasing bone length, increasing bone microarchitecture, and/or combinations thereof.

The term “stunting” as used herein is defined as height- or length-for-age that is less than two standard deviations below the mean for reference children.

The methods of promoting bone health in a moderately malnourished individual according to the present disclosure are based on the discovery that treating a moderately malnourished individual with a nutritional composition having a particular blend of carbohydrates increases bone density and/or content, increases bone length, improves bone microarchitecture, or a combination thereof.

The methods of promoting bone health in a moderately malnourished individual according to the present disclosure comprise the administration of a nutritional composition to the moderately malnourished individual. The nutritional composition administered to the moderately malnourished individual comprises a carbohydrate blend. In accordance with the present disclosure, the nutritional composition administered to the moderately malnourished individual may comprise one or more of a protein and a fat, in addition to the carbohydrate blend.

The carbohydrate blend comprises a source of at least one carbohydrate that provides rapidly available glucose, a source of at least one carbohydrate that provides slowly available glucose, and a source of at least one non-digestible carbohydrate or resistant starch. Carbohydrates that provide rapidly available glucose are rapidly absorbed in the duodenum and proximal regions of the small intestine leading to a rapid elevation of blood glucose and usually a subsequent episode of hypoglycemia. Carbohydrates that provide slowly available glucose are steadily but completely digested resulting in prolonged glucose release from the lumen of the small intestine into the blood stream. Non-digestible carbohydrates or resistant starches are carbohydrates or factions thereof that are not digested in the upper gastrointestinal tract but are fermented in the large intestine by the gut microflora, producing short chain fatty acids that provide additional energy to the body.

The terms “rapidly available glucose” and “slowly available glucose” as used herein reflect the rate at which glucose becomes available for absorption in the human small intestine according to the in vitro method developed by Englyst et al. (Am J Clin Nutr (1999), Vol. 69, pp 448-454), which is incorporated by reference herein. This in vitro method characterizes dietary carbohydrates with regard to their chemical composition and likely gastrointestinal fate. The glycemic carbohydrate fraction that is available for absorption in the small intestine is measured as the sum of sugars and starch (including maltodextrins) and excludes resistant starch. The Englyst method determines rapidly available glucose, slowly available glucose, and starch fractions by measuring the amount of glucose released from a carbohydrate or carbohydrate source during timed incubation (20 minutes and 120 minutes) with digestive enzymes under standardized conditions. In accordance with the Englyst method, for a carbohydrate or carbohydrate source the amount of glucose measured at 20 minutes (G₂₀) represents “rapidly available glucose,” whereas the difference between the amount of glucose measured at 120 minutes (G₁₂₀) and the G₂₀ value (i.e., G₁₂₀-G₂₀) represents “slowly available glucose.” The Englyst method also enables the calculation of: (i) rapidly digestible starch, which contributes to the amount of rapidly available glucose; (ii) slowly digestible starch, which contributes to the amount of slowly available glucose; (iii) total starch; and (iv) resistant starch.

In accordance with the present disclosure, the carbohydrate blend comprises a source of at least one carbohydrate that provides rapidly available glucose. In accordance with the present disclosure, the source of at least one carbohydrate that provides rapidly available glucose comprises one or more of: (i) a monosaccharide; (ii) a glucose unit and a fructose unit joined by an α-1, β-2 glycosidic linkage; (iii) a glucose unit and a galactose unit joined by a β (1,4) glycosidic linkage; (iv) glucose units joined by α (1,4) glycosidic linkages; (v) glucose units joined by α (1,6) glycosidic linkages; and (vi) an oligosaccharide having a random mixture of α (1,2), α (1,3), α (1,4), and α (1,6) glycosidic linkages.

In accordance with the present disclosure, the source of at least one carbohydrate that provides rapidly available glucose in the carbohydrate blend comprises a monosaccharide. Exemplary monosaccharides suitable for use in the carbohydrate blend to provide rapidly available glucose include, but are not limited to, glucose, fructose, tagatose, galactose, mannose, and ribose.

In accordance with the present disclosure, the source of at least one carbohydrate that provides rapidly available glucose in the carbohydrate blend comprises a glucose unit and a fructose unit joined by an α-1, β-2 glycosidic linkage. One example of a carbohydrate that includes a glucose unit and a fructose unit joined by an α-1, β-2 glycosidic linkage is sucrose.

In accordance with the present disclosure, the source of at least one carbohydrate that provides rapidly available glucose in the carbohydrate blend comprises a galactose unit and a glucose unit joined by a β (1,4) glycosidic linkage. One example of a carbohydrate that includes a galactose unit and a glucose unit joined by a β (1,4) glycosidic linkage is lactose.

In accordance with the present disclosure, the source of at least one carbohydrate that provides rapidly available glucose in the carbohydrate blend comprises glucose units joined by α (1,4) glycosidic linkages. Exemplary carbohydrates or carbohydrate sources having glucose units joined by α (1,4) glycosidic linkages include, but are not limited to, maltose, maltodextrin, and starch.

In accordance with the present disclosure, the source of at least one carbohydrate that provides rapidly available glucose in the carbohydrate blend comprises glucose units joined by α (1,6) glycosidic linkages. One example of a carbohydrate having glucose units joined by α (1,6) glycosidic linkages is isomaltose.

In accordance with the present disclosure, the source of at least one carbohydrate providing rapidly available glucose in the carbohydrate blend comprises oligosaccharides having a random mixture of α (1,2), α (1,3), α (1,4), and α (1,6) glycosidic linkages. One example of a source of carbohydrate that includes oligosaccharides having a random mixture of α (1,2), α (1,3), α (1,4), and α (1,6) glycosidic linkages is isomalto-oligosaccharides. Suitable isomalto-oligosaccharides that provide rapidly available glucose include mixtures of oligosaccharides with a degree of polymerization (DP) of 3 or greater including, but not limited to, isomaltose, panose, maltotetraose, isomaltotriose, isomaltotetraose, maltopentaose, isomaltopentaose, maltohexaose, isomaltohexaose, maltoheptaose, isomaltoheptaose, maltooctaose, isomaltooctaose, maltononaose, and isomaltononaose.

In accordance with the present disclosure, the carbohydrate blend comprises a source of at least one carbohydrate that provides slowly available glucose. In embodiments, the source of at least one carbohydrate that provides slowly available glucose comprises one or more of: (i) a glucose unit and a fructose unit joined by an α (1,6) glycosidic linkage; (ii) two glucose units joined by an α (1,1) glycosidic linkage; (iii) a glucose unit and a fructose unit joined by an α (1,5) glycosidic linkage; and (iv) an oligosaccharide having alternating α (1,3) and α (1,6) glycosidic linkages.

In accordance with the present disclosure, the source of at least one carbohydrate providing slowly available glucose in the carbohydrate blend comprises a glucose unit and a fructose unit joined by an α (1,6) glycosidic linkage. One example of a carbohydrate having a glucose unit and a fructose unit joined by an α (1,6) glycosidic linkage is isomaltulose.

In accordance with the present disclosure, the source of at least one carbohydrate providing slowly available glucose in the carbohydrate blend comprises two glucose units joined by an α (1,1) glycosidic linkage. One example of a carbohydrate having two glucose units joined by an α (1,1) glycosidic linkage is trehalose.

In accordance with the present disclosure, the source of at least one carbohydrate providing slowly available glucose in the carbohydrate blend comprises a glucose unit and a fructose unit joined by an α (1,5) glycosidic linkage. One example of a carbohydrate having a glucose unit and a fructose unit joined by an α (1,5) glycosidic linkage is leucrose. Leucrose is a disaccharide that is present in sucromalt.

In accordance with the present disclosure, the source of at least one carbohydrate that provides slowly available glucose in the carbohydrate blend comprises oligosaccharides having alternating α (1,3) and α (1,6) glycosidic linkages. One example of a source of carbohydrate that includes oligosaccharides having alternating α (1,3) and α (1,6) glycosidic linkages is sucromalt.

In accordance with the present disclosure, the carbohydrate blend comprises a source of at least one non-digestible carbohydrate or resistant starch. In embodiments, the source of at least one non-digestible carbohydrate or resistant starch comprises one or more of: (i) oligosaccharides having a random mixture of α (1,2), α (1,3), α (1,4), and β glycosidic linkages; (ii) saccharides having linear chains of 2 to 60 fructose units joined by α (2,1) glycosidic linkages or fructose polymers joined by β (2,1) glycosidic linkages; and (iii) oligosaccharides having a random mixture of α (1,2), α (1,3), α (1,4), and α (1,6) glycosidic linkages.

In accordance with the present disclosure, the source of at least one non-digestible carbohydrate or resistant starch (including maltodextrin) comprises oligosaccharides having a random mixture of α (1,2), α (1,3), α (1,4), and β glycosidic linkages. One example of a source of carbohydrate that includes oligosaccharides having a random mixture of α (1,2), a (1,3), α (1,4), and β glycosidic linkages is resistant maltodextrin. The mixture of oligosaccharides making up resistant maltodextrin are produced by pyrolysis and enzymatic hydrolysis of starch (e.g., corn, wheat, rice, potato) and have a molecular weight of about 2,000 Daltons. Examples of commercially available resistant maltodextrin include NUTRIOSE resistant maltodextrin from Roquette America, Inc. (Geneva, Ill.) and FIBERSOL digestion resistant maltodextrin from ADM/Matsutani LLC (Itasca, Ill.).

In accordance with the present disclosure, the source of at least one non-digestible carbohydrate or resistant starch comprises saccharides having linear chains of 2 to 60 fructose units or fructose polymers joined by β (2,1) glycosidic linkages. Exemplary carbohydrates and carbohydrate sources that include saccharides having linear chains of 2 to 60 fructose units or fructose polymers joined by β (2,1) glycosidic linkages include, but are not limited to, inulin and fructooligosaccharide.

In accordance with the present disclosure, the source of at least one non-digestible carbohydrate or resistant starch comprises oligosaccharides having a random mixture of α (1,2), α (1,3), α (1,4), and α (1,6) glycosidic linkages. One example of a source of carbohydrate that includes oligosaccharides having a random mixture of α (1,2), α (1,3), α (1,4), and α (1,6) glycosidic linkages is isomalto-oligosaccharides. The isomalto-oligosaccharides may be any one or more of the isomalto-oligosaccharides previously described herein.

In accordance with the present disclosure, the carbohydrate blend comprises: (i) a source of at least one carbohydrate that provides rapidly available glucose selected from one or more of glucose, fructose, galactose, mannose, ribose, sucrose, lactose, maltose, isomaltose, maltodextrin, starch, or isomalto-oligosaccharides; (ii) a source of at least one carbohydrate that provides slowly available glucose selected from one or more of isomaltulose, trehalose, leucrose, or sucromalt; and (iii) a source of at least one non-digestible or resistant starch selected from one or more of resistant starch, fructooligosaccharides, inulin, or isomalto-oligosaccharides. In embodiments, the carbohydrate blend comprises: (i) a source of at least one carbohydrate that provides rapidly available glucose selected from one or more of maltodextrin, isomaltose, or isomalto-oligosaccharides; (ii) a source of at least one carbohydrate that provides slowly available glucose selected from one or more of isomaltulose, sucromalt, trehalose, or leucrose; and (iii) a source of at least one non-digestible or resistant starch selected from one or more of resistant starch, fructooligosaccharides, inulin, or isomalto-oligosaccharides. In embodiments, the carbohydrate blend comprises: (i) a source of at least one carbohydrate that provides rapidly available glucose selected from one or more of maltodextrin or isomalto-oligosaccharides; (ii) a source of at least one carbohydrate that provides slowly available glucose selected from one or more of isomaltulose or sucromalt; and (iii) a source of at least one non-digestible or resistant starch selected from one or more of resistant starch, fructooligosaccharides, inulin, or isomalto-oligosaccharides.

Certain sources of carbohydrates used in the carbohydrate blend may include carbohydrates or fractions thereof that provide more than one category of glucose availability (i.e., rapidly available glucose, slowly available glucose, and non-available glucose (e.g., non-digestible carbohydrate or resistant starch)). Thus, in accordance with the methods of the present disclosure, a source of carbohydrate in the carbohydrate blend can include one or more of a carbohydrate that provides rapidly available glucose, a carbohydrate the provides slowly available glucose, and a non-digestible carbohydrate or resistant starch. For example, a source of carbohydrate that includes a carbohydrate that provides rapidly available glucose and a non-digestible carbohydrate or resistant starch (e.g., through a fiber fraction, which is a non-digestible carbohydrate or resistant starch) is isomalto-oligosaccharides. An example of a source of carbohydrate that includes a carbohydrate that provides rapidly available glucose and a carbohydrate that provides slowly available glucose is sucromalt. Accordingly, a single source of carbohydrate may be used in the methods of the present disclosure to provide one or more than one of a carbohydrate that provides rapidly available glucose, a carbohydrate that provides slowly available glucose, and a non-digestible carbohydrate or resistant starch.

In accordance with the present disclosure, the source of at least one carbohydrate of the carbohydrate blend that provides rapidly available glucose provides from about 4% to about 71%, including from about 10% to about 70%, including from about 13% to 64%, and including from about 16% to about 60% of the total calories supplied by carbohydrates in the nutritional composition, the source of at least one carbohydrate of the carbohydrate blend that provides slowly available glucose provides from about 25% to about 86%, including from about 30% to about 80%, including from about 35% to about 75%, and including from about 40% to about 70% of the total calories supplied by carbohydrates in the nutritional composition, and the source of at least one non-digestible carbohydrate or resistant starch of the carbohydrate blend provides from about 3% to about 20%, including from about 5% to about 18%, including from about 7% to about 15%, and including from about 8% to about 12% of the total calories supplied by carbohydrates in the nutritional composition. A carbohydrate blend having such a distribution of carbohydrates with the specified glucose availability accelerates the development of healthy catch-up growth by establishing and maintaining a more balanced use of carbohydrates and fats as energy sources.

In accordance with the present disclosure, the ratio based on the percentage of total calories of slowly digestible carbohydrate to non-digestible carbohydrate or resistant starch ranges from about 29:1 to about 5:4, the ratio non-digestible carbohydrate or resistant starch to rapidly digestible carbohydrate ranges from about 5:1 to about 1:24, and the ratio of slowly digestible carbohydrate to rapidly digestible carbohydrate ranges from about 22:1 to about 1:18.

As previously mentioned, in accordance with the methods of the present disclosure, the nutritional composition administered to the moderately malnourished individual comprises one or more of a protein and a fat, in addition to the carbohydrate blend. In accordance with the method of the present disclosure, the nutritional composition administered to the moderately malnourished individual comprises a protein, a fat, and a carbohydrate blend as previously described.

As previously mentioned, a nutritional composition for use in treating malnourishment in a moderately malnourished individual, comprises one or more of a protein and a fat, in addition to a carbohydrate blend as previously described.

In addition, in use of a nutritional composition for the manufacture of a medicament for treating malnourishment in a moderately malnourished individual, the nutritional composition comprises one or more of a protein and a fat, in addition to a carbohydrate blend as previously described.

The amount of carbohydrates in the nutritional composition will typically range from about 5% to about 75%, including from about 15% to about 70%, including from about 30% to about 65%, by weight of the nutritional composition, on a dry weight basis. When present, the amount of fat in the nutritional composition will typically range from about 1% to about 30%, including from about 2% to about 15%, and also including from about 3% to about 10%, by weight of the nutritional composition, on a dry weight basis. When present, the amount of protein in the nutritional composition will typically range from about 0.5% to about 30%, including from about 1% to about 25%, and also including from about 10% to about 20%, by weight of the nutritional composition, on a dry weight basis.

The amount of any or all of the carbohydrates, fats, and proteins in any of the nutritional compositions described herein may also be characterized as a percentage of total calories in the nutritional composition as set forth in the following table. The macronutrients for nutritional compositions suitable for use in accordance with the methods of the present disclosure are most typically formulated within any of the caloric ranges (embodiments A-F) described in the following table (each numerical value is preceded by the term “about”).

TABLE 1
Exemplary macronutrient profiles of nutritional compositions

| | Embodiment A | Embodiment B | Embodiment C |
|---|---|---|---|
| Nutrient | (% Total Cal.) | (% Total Cal.) | (% Total Cal.) |
| Carbohydrate | 1-98 | 2-96 | 10-75 |
| Protein | 1-98 | 2-96 | 5-70 |
| Fat | 1-98 | 2-96 | 20-85 |
| | Embodiment D | Embodiment E | Embodiment F |
| | (% Total Cal.) | (% Total Cal.) | (% Total Cal.) |
| Carbohydrate | 30-50 | 25-50 | 25-50 |
| Protein | 15-35 | 10-30 | 5-30 |
| Fat | 35-55 | 1-20 | 2-20 |

The nutritional composition of the present disclosure may comprise a fat or a source of fat. The fat or source of fat used in the nutritional composition may be derived from various sources including, but not limited to, plants, animals, and combinations thereof.

Sources of fat that are suitable for use in the nutritional composition include, but are not limited to, coconut oil, fractionated coconut oil, soy oil, high oleic soy oil, corn oil, olive oil, safflower oil, high oleic safflower oil, medium chain triglyceride oil (MCT oil), high gamma linolenic (GLA) safflower oil, sunflower oil, high oleic sunflower oil, palm oil, palm kernel oil, palm olein, canola oil, high oleic canola oil, marine oils, fish oils (e.g., tuna oil), algal oils, borage oil, cottonseed oil, fungal oils, eicosapentaenoic acid (EPA), docosahexaenoic acid (DHA), arachidonic acid (ARA), conjugated linoleic acid (CLA), alpha-linolenic acid, interesterified oils, transesterified oils, structured lipids, and combinations thereof.

Generally, the fat or source of fat used in the nutritional composition provides fatty acids needed both as an energy source and for the healthy development of the individual. The source of fat typically comprises triglycerides, although the source of fat may also comprise diglycerides, monoglycerides, phospholipids (e.g., lecithin) and free fatty acids. Fatty acids provided by the source of fat in the nutritional composition include, but are not limited to, capric acid, lauric acid, myristic acid, palmitic acid, palmitoleic acid, stearic acid, oleic acid, linoleic acid, alpha-linolenic acid, ARA, EPA, DHA, and combinations thereof. The nutritional composition administered in accordance with the methods of the present disclosure may include any individual source of fat or combination of the various sources of fat listed above.

The nutritional composition of the present disclosure may comprise a protein or a source of protein. The protein or source of protein used in the nutritional composition may be a plant-based protein, an animal-based protein, a milk-based protein, and combinations of the foregoing. The protein may be intact, partially hydrolyzed (i.e., a degree of hydrolysis of less than 20%), or extensively hydrolyzed (i.e., a degree of hydrolysis of at least 20%). The protein may also include at least one free amino acid.

Plant-based proteins suitable for use in the nutritional composition include, but are not limited to, soy protein, pea protein, rice protein, and potato protein. Animal-based proteins suitable for use in the nutritional composition include, but are not limited to, poultry protein (e.g., chicken protein), collagen, fish protein, ovine protein, porcine protein, and bovine protein. Milk-based proteins suitable for use in the nutritional composition include, but are not limited to, whole cow's milk, partially or completely defatted milk, milk protein concentrates, milk protein isolates, nonfat dry milk, condensed skim milk, whey protein concentrates, whey protein isolates, acid caseins, sodium caseinates, calcium caseinates, and potassium caseinates. The source of protein may be certified organic (e.g., USDA organic) or non-organic, and also may be non-genetically modified (non-GMO) or genetically modified. The nutritional composition for use in accordance with the methods of the present disclosure may include any individual source of protein or combination of the various sources of protein listed above.

In addition, the source of protein in the nutritional composition can also include one or more free amino acids. Free amino acids that may be used in the nutritional composition include, but are not limited to, L-lysine, L-tryptophan, L-glutamine, L-tyrosine, L-methionine, L-cysteine, taurine, L-arginine, L-leucine, L-isoleucine, L-valine, and L-carnitine.

The nutritional composition of the present disclosure may comprise vitamins and minerals. In accordance with the present disclosure, the nutritional composition comprises at least one of vitamin A, vitamin D, vitamin E, vitamin K, thiamine, riboflavin, pyridoxine, vitamin B₁₂, niacin, folic acid, pantothenic acid, biotin, vitamin C, choline, and inositol. In embodiments, the nutritional composition comprises at least one of calcium, phosphorus, magnesium, iron, zinc, manganese, copper, sodium, potassium, molybdenum, chromium, selenium, chloride, and iodine.

The nutritional composition of the present disclosure may also include a variety of optional ingredients that may modify the physical, chemical, or processing characteristics of the nutritional composition, or merely serve as additional nutritional component. In accordance with the present disclosure, the nutritional composition may comprise one or more of a probiotic, a nucleotide, a nucleoside, and a carotenoid (e.g., lutein, beta-carotene, lycopene, zeaxanthin).

The nutritional composition of the present disclosure may have a caloric density tailored to the nutritional needs of the ultimate user. In embodiments of the present disclosure, the nutritional composition has a caloric density of at least 1 kcal/mL. In accordance with the present disclosure, the nutritional composition has a caloric density of 1 kcal/mL to 2 kcal/mL, including from 1.1 kcal/mL to 2 kcal/mL, from 1.15 kcal/mL to 1.9 kcal/mL, from 1.2 kcal/mL to 1.8 kcal/mL, and also including from 1.25 kcal/mL to 1.5 kcal/mL.

The nutritional composition of the present disclosure may be formulated and administered in any known or otherwise suitable oral product form. Any solid, liquid, semi-solid, and semi-liquid, or powder product form, including combinations or variations thereof, are suitable for use herein, provided that such forms allow for safe and effective oral delivery to the individual of the essential ingredients disclosed herein.

The nutritional composition may be in any product form comprising the ingredients described herein, and which is safe and effective for oral administration. The nutritional composition may be formulated to include only the ingredients described herein, or may be modified with optional ingredients to form a number of different product forms.

In accordance with the present disclosure, the nutritional composition administered in accordance with the methods of the present disclosure is formulated as a liquid nutritional composition. Liquid nutritional compositions include both concentrated and ready-to-feed nutritional liquids. These nutritional liquids are most typically formulated as suspensions or emulsions, although other liquid forms are within the scope of the present disclosure. Nutritional compositions in the form of emulsions suitable for use may be aqueous emulsions comprising proteins, fats, and carbohydrates. These emulsions are generally flowable or drinkable liquids at from about 1° C. to about 25° C. and are typically in the form of oil-in-water, water-in-oil, or complex aqueous emulsions, although such emulsions are most typically in the form of oil-in-water emulsions having a continuous aqueous phase and a discontinuous oil phase.

The nutritional liquid may be and typically is shelf stable. The nutritional liquid typically contains up to about 95% by weight of water, including from about 50% to about 95%, also including from about 60% to about 90%, and also including from about 70% to about 85%, of water by weight of the nutritional liquid. The nutritional liquid may have a variety of product densities, but most typically have a density greater than about 1.03 g/mL, including greater than about 1.04 g/mL, including greater than about 1.055 g/mL, including from about 1.06 g/mL to about 1.12 g/mL, and also including from about 1.085 g/mL to about 1.10 g/mL. The nutritional liquid may have a pH ranging from about 2.5 to about 8, but is most advantageously in a range of from about 4.5 to about 7.5, including from about 5.5 to about 7.3, including from about 6.2 to about 7.2.

Although the serving size for the nutritional liquid can vary depending upon a number of variables, a typical serving size is generally at least about 1 mL, or even at least about 2 mL, or even at least about 5 mL, or even at least about 10 mL, or even at least about 25 mL, including ranges from about 1 mL to about 360 mL, including from about 30 mL to about 250 mL, and including from about 60 mL to about 240 mL.

In accordance with the present disclosure, the nutritional composition of the present disclosure can also be formulated as a nutritional solid. The nutritional solids may be in any solid form but are typically in the form of flowable or substantially flowable particulate compositions, or at least particulate compositions. Particularly suitable nutritional solid product forms include spray dried, agglomerated, and/or dryblended powder compositions. The compositions can easily be scooped and measured with a spoon or similar other device, and can easily be reconstituted with a suitable aqueous liquid, typically water, to form a nutritional composition for immediate oral or enteral use. In this context, “immediate” use generally means within about 48 hours, most typically within about 24 hours, preferably right after reconstitution.

As previously mentioned, the methods of promoting bone health in a moderately malnourished individual during a period of catch-up growth according to the present disclosure comprise treating the moderately malnourished individual by administering a nutritional composition to the moderately malnourished individual, including during the period of catch-up growth. The nutritional composition comprises a carbohydrate blend as previously described. Any of the various nutritional compositions described herein can be administered to the moderately malnourished individual in accordance with the methods of the present disclosure.

As previously mentioned, a nutritional composition for use in treating malnourishment in a moderately malnourished individual, comprises a carbohydrate blend as previously described.

In addition, in use of a nutritional composition for the manufacture of a medicament for treating malnourishment in a moderately malnourished individual, the nutritional composition comprises a carbohydrate blend as previously described.

In accordance with the present disclosure, the moderately malnourished individual may be an infant, a preterm infant, a toddler, a child. In embodiments of the method of the present disclosure, the moderately malnourished individual suffers from one or more of stunting and wasting. As briefly mentioned above, stunting is characterized by a low height-for-age, such as a height-for-age z-score of less than −2 based on the WHO child growth standards median. Similarly, wasting is characterized by a low weight-for-height, such as a weight-for-height z-score of less than −2 based on the WHO child growth standards median. Stunting and wasting may be caused by a number of factors including, but not limited to, malnutrition, undernutrition, poor gut health, infectious disease and compromised immune function.

In accordance with the methods of the present disclosure, the moderately malnourished individual is an individual who suffers from stunting, and the stunting is caused by one or more of malnutrition, undernutrition, poor gut health, infectious disease, and compromised immune function.

In accordance with the methods of the present disclosure, the moderately malnourished individual is an individual who suffers from wasting, and the wasting is caused by one or more of malnutrition, undernutrition, poor gut health, infectious disease, and compromised immune function.

The methods of promoting bone health in a moderately malnourished individual according to the present disclosure are applied to prevent or treat stunting in individuals having decreased bone health, decreased bone density and/or content, decreased bone length and/or microarchitecture, or one or more combinations thereof.

The methods of promoting bone health in a moderately malnourished individual according to the present disclosure are applied to prevent or treat wasting in individuals having decreased bone health, decreased bone density and/or content, decreased bone length and/or microarchitecture, or one or more combinations thereof.

The methods of promoting bone health in a moderately malnourished individual according to the present disclosure are applied to prevent or treat osteoporosis in individuals having decreased bone health, decreased bone density and/or content, decreased bone length and/or microarchitecture, or one or more combinations thereof. Treating individuals, such as preterm infants, infants. toddlers, or children, according to methods of the present disclosure yields improved bone health in such individuals. Improved bone health in individuals further yields a decreased risk of individuals developing osteoporosis later in life. As such, treating individuals according to the methods of the present disclosure aids individuals in preventing osteoporosis later in life.

The methods of promoting bone health in a moderately malnourished individual according to the present disclosure are applied to prevent or treat osteomalacia in individuals having decreased bone health, decreased bone density and/or content, decreased bone length and/or microarchitecture, or one or more combinations thereof.

The methods of promoting bone health in a moderately malnourished individual according to the present disclosure are applied to prevent or treat rickets in individuals having decreased bone health, decreased bone density and/or content, decreased bone length and/or microarchitecture, or one or more combinations thereof.

In accordance with the methods of the present disclosure, administration of a nutritional composition having a carbohydrate blend as described herein improves bone health by at least one of: increasing bone density and/or content, increasing bone length, improving bone microarchitecture, and one or more combination thereof.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

To the extent that the term “includes” or “including” is used in the description or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use.

The methods of the present disclosure can comprise, consist of, or consist essentially of the essential elements of the disclosure as described herein, as well as any additional or optional element described herein, or which is otherwise useful in nutritional applications.

All percentages, parts, and ratios as used herein are by weight of the total composition unless otherwise specified. All such weights as they pertain to listed ingredients are based on the active level and, therefore, do not include solvents, by-products, or other components that may be included in commercially available materials, unless otherwise specified.

All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, and 10) contained within the range.

Any combination of method or process steps as used herein may be performed in any order, unless otherwise specified or clearly implied to the contrary by the context in which the referenced combination is made.

### EXAMPLES

The following example is presented to provide a better understanding of the methods of the present disclosure. The examples are for purposes of illustration only and are not intended to limit the scope of the present disclosure.

Example 1—A pre-clinical study was conducted to evaluate the effect of chronic administration of rapidly or slowly digestible carbohydrate blends on bone health and the associated outcomes using an animal model of growth retardation or nutritional dwarfing (ND).

The nutritional dwarfing model is based on developing a nutritional stress in weanling male rats placed on restricted intake (30% of normal intake) of the control diet for four weeks. The restriction period was followed by another four weeks with full access to the experimental diets.

As discussed above, “RDC” refers to a diet with rapidly digestible carbohydrate diet and “SDC” refers to a diet with the inventive carbohydrate blend. “BMC” refers to bone mineral content; “BMD” refers to bone mineral density; “BV/TV” refers to the percent bone volume; “TbS” refers to trabecular space; and “TbN” refers to trabecular number. Results are presented as mean.

During the restriction period, animals were divided into two groups. Non-restricted Rats (NR group) were fed with a standard rodent diet (AIN93M) ad libitum during the entire study period. Restricted Rats (RR group) received 70% of the amount of food consumed by the NR group. After the four-week food restriction period, the RR group was fed ad libitum with different humanized experimental diets (RDC group and SDC group) for four weeks (the re-feeding period). The RDC and SDC diets were designed to be provide similar amount of protein, fat and carbohydrate. However, it should be noted that the RDC diet predominantly contains sources of carbohydrate that provide rapidly available glucose while the SDC diet contains a carbohydrate blend that includes a source of carbohydrate that provides rapidly available glucose, a source of carbohydrate that provides slowly available glucose, and a source of non-digestible carbohydrate or resistant starch in accordance with the present invention. The AIN93G diet is a purified rodent diet structured to provide nutrients in concentrations required just to maintain adult rat or mouse populations. The compositions of the diets are provided in Table 1 below.

TABLE 1
Composition of Diets

| | AIN93G | RDC | SDC |
|---|---|---|---|
| Macronutrients | Diet | Diet | Diet |
| Carbohydrates (CHO) (g/100 g diet) | 64.59 | 67 | 67 |
| Sucrose (g/100 g CHO) | 14.37 | 32 | |
| Isomaltulose (g/100 g CHO) | | | 26.4 |
| Sucromalt (g/100 g CHO) | | | 22.1 |
| Cornstarch (g/100 g CHO) | 59.48 | | |
| Maltodextrins (g/100 g CHO) | 18.97 | 64 | 23 |
| IMOs (g/100 g CHO) | | | 11.5 |
| Resistant starch (g/100 g CHO) | | | 10 |
| FOS (g/100 g CHO) | | 4 | |
| Inulin: FOS (g/100 g CHO) | | | 7 |
| Cellulose (g/100 g CHO) | 5 | | |
| Protein (g/100 g diet) | 18.30 | 15.03 | 15.03 |
| Fat (g/100 g diet) | 7.00 | 9.80 | 9.80 |

RDC: diet with rapidly digestible CHO; SDC: diet with slowly digestible CHO; CHO: carbohydrates; IMOs = isomalto-oliogsaccharides; FOS = fructooligosaccharides; Inulin: FOS = 1:1 mixture of inulin and fructooligosaccharides.

After killing the animals, tibias and lumbar vertebrae (LV2-5) were carefully removed from each animal. The isolated appendicular (tibia) and the axial (lumbar vertebrae) bones were analyzed utilizing a DXA and micro-CT technical approach to determine key bone mass and structural markers related to bone quantity and quality.

To analyze bone quantity, tibia length as well as bone mineral content (BMC) and density (BMD) in the tibia and vertebrae were analyzed by dual energy X-ray absorptiometry using a DXA densitometer (UltraFocus DEXA, Faxitron, Tucson, USA). DEXA scan is a commonly used to analyze bone health in clinical practice. Tibial bone length was significantly much shorter in stunted rats (RR group) than in the control group (NR group) at 7 weeks of age, but after dietary deficiency resolved (at 7-12 weeks of age) an accelerated increase in tibial length was observed in both experimental groups (RDC and SDC). During the catch-up growth period, tibial length of the RDC group was increased by around 25%, reaching similar values compared with NR group at 12 week of age. However, the stunted rats which were fed a second time with the SDC diet showed higher bone longitudinal rate, promoting an increase around 38% in the tibial length. More interestingly, the tibial length in the SDC group exceeded the bone length found in the NR and RDC groups by about 13%.

To analyze the trabecular bone quality, percent bone volume (BV/TV), trabecular thickness (Tb/Th), and number (TbN) in tibia and vertebrae were analyzed by microcomputed tomography (micro-CT; Scanco, Medical, Basserdorf, Switzerland). A person having ordinary skill in the art would understand micro-CT has become the “gold-standard” for the evaluation of bone morphology and micro-architecture in rodents ex vivo, as it enables direct 3D measurement of trabecular morphology.

In the present study, the level of food restriction imposed was severe enough to decrease the normal bone development in restricted animals.

At the end of the food restriction period the data showed that bone quantity related markers, BMC and BMD, were significantly lower than in restricted rats as compared to non-restricted rats. The bone quality was similarly affected by the restriction period. In this way, BV/TV, TbTh, and TbTn were lower in the restricted rats than in non-restricted rats, while TbS was greater (Table 2). There results indicate that in the critical period for obtaining an adequate bone peak mass, nutritional stunting process resulted in lower bone volume and a more fragile bone architecture in the undernourished rats. The negative effects on bone status by the food restriction period were partially recovered during the re-feeding period. However, the recovery degree was significantly different depending on the composition of the carbohydrate blend used in RDC vs. SDC (Table 2).

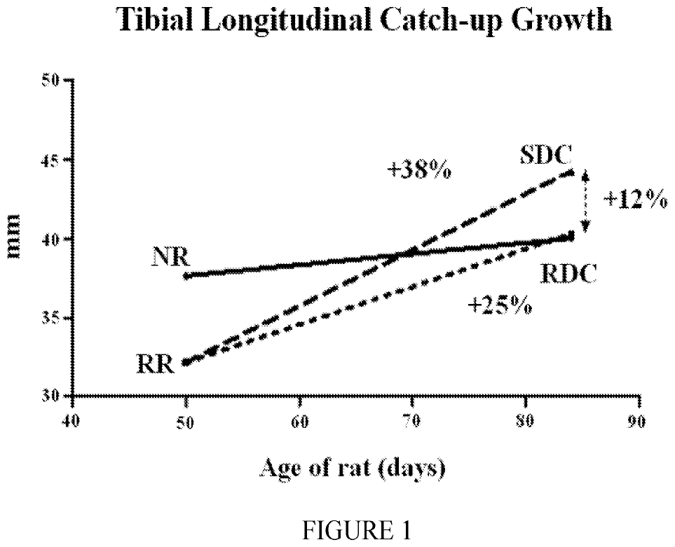

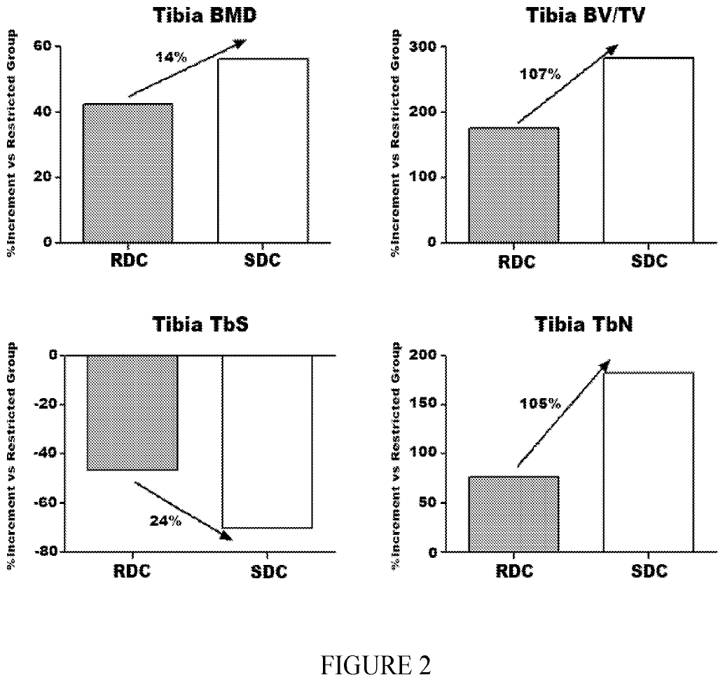

Animals fed a SDC diet showed a significantly higher BMD (approximately 10%) than the animals fed on the RDC diet. This increase is essential to achieve an optimal bone health and reduce the risk of fracture later in life. In fact, it has been described that a 10% increase in peak bone mineral mass of children could reduce the risk of fracture associated with osteoporosis by 50%. Further, the animals of the SDC group recovered more bone regarding the values obtained at the end of the restriction period than the animals of the RDC group (See FIGS. 1-2).

TABLE 2
Changes in bone quantity and quality parameters after restriction period
(NR and RR groups) and re-feeding period (RDC and SDC groups).

| Groups | | | |
|---|---|---|---|
| NR | RR | Re-feeding | |
| Diet | | | |
| AIN93G | AIN93G | RDC | SDC |

TIBIA

| | | | | | |
|---|---|---|---|---|---|
| Length | (mm) | 37.66 ± 0.66 | 32.17 ± 1.16 | 40.31 ± 1.69 | 44.25 ± 2.92 |
| Quantity | BMC (g) | 0.158 ± 0.003 | 0.101 ± 0.004″ | 0.290 ± 0.015 | 0.301 ± 0.020 |
| | BMD (mg/cm²) | 116.7 ± 1.4 | 102.8 ± 0.2^{#} | 146.3 ± 3.6 | 160.4 ± 6.3* |
| Quality | BV/TV (%) | 0.120 ± 0.011 | 0.043 ± 0.007′ | 0.118 ± 0.005 | 0.164 ± 0.009* |
| | TbTh (mm) | 0.05110.001 | 0.050 ± 0.001 | 0.054 ± 0.001 | 0.05110.001* |
| | TbS (mm) | 0.2750.017 | 0.698 ± 0.095′ | 0.374 ± 0.028 | 0.208 ± 0.013* |
| | TbN (/mm) | 3.33 ± 0.27 | 1.57 ± 0.21′ | 2.77 ± 0.18 | 4.43 ± 0.31* |

LUMBAR VERTEBRA

| | | | | | |
|---|---|---|---|---|---|
| Quantity | BMC (g) | 0.208 ± 0.010 | 0.132 ± 0.006′ | 0.583 ± 0.042 | 0.675 ± 0.040* |
| | BMD (mg/cm²) | 141.8 ± 2.9 | 114.9 ± 2.3″ | 180.1 ± 7.7 | 202.8 ± 6.4* |
| Quality | BV/TV (%) | 0.28210.011 | 0.23710.010′ | 0.265 ± 0.003 | 0.307 ± 0.014* |
| | TbTh (mm) | 0.07710.001 | 0.06610.002′ | 0.07310.001 | 0.072 ± 0.001 |
| | TbS (mm) | 0.244 ± 0.008 | 0.258 ± 0.004 | 0.251 ± 0.004 | 0.226 ± 0.008* |
| | TbN (hmm) | 3.78 ± 0.10 | 3.59 ± 0.05 | 3.67 ± 0.05 | 4.06 ± 0.12* |

NR: non-restricted group; RR: restricted group; RDC: diet with rapidly digestible CHO diet; SDC: diet with slowly digestible CHO; BMC: bone mineral content; BMD: bone mineral density; BV/TV: percent bone volume; TbTh: trabecular thickness; TbS: trabecular space; TbN: trabecular number.
Results are presented as mean ± SEM.)
p < 0.05 compared with NR group;
*p < 0.05 compared with RDC group.

In addition, the SDC diet improved the bone structural parameters. Tibia and lumbar vertebrae of the SDC group showed a significant increase in trabecular BV/TV and TbN, concomitant with a significant decrease in TbS in comparison with those of the RDC group (Table 2). Compared with the restricted group (FIG. 1-2), the percentage of changes in trabecular parameters were markedly higher in the SDC group than in the RDC group, changing substantially to better retain architectural bone integrity.

The results demonstrate the intake of the carbohydrate blend based on the SDC diet in accordance with the present invention in the growth period improve bone accretion/development in the catch-up period after a food restriction period. Overall, this study suggests that this carbohydrate blend would be helpful to children with stunted growth for age by nutritional restriction to obtain the optimal development of trabecular morphology during the period of bone accrual and may confer bone strength advantages not only in youth but also throughout the life span.

The study results indicate that diets containing a carbohydrate blend of sources of rapidly available glucose, slowly available glucose, and non-digestible carbohydrates or resistant starch, as present in the SDC diet in accordance with the present invention, can reduce the development of osteoporosis, osteomalacia, and rickets, as well as maintain adequate bone mass, strength and microarchitecture in moderately malnourished individuals.

While the present disclosure has been illustrated by the description of embodiments thereof, and while the embodiments have been described in considerable detail, it is not the intention of the applicant to restrict or in any way limit the scope of the appended claims to such detail. Additional advantages and modifications will readily appear to those skilled in the art. Therefore, the present disclosure, in its broader aspects, is not limited to the specific details, the representative compositions or formulations, and illustrative examples shown and described. Accordingly, departures may be made from such details without departing from the spirit or scope of the applicant's general disclosure herein.

1.-15. (canceled)

16. A method of promoting bone health in a moderately malnourished individual, the method comprising:
administering to the moderately malnourished individual a nutritional composition comprising a carbohydrate blend of:
(i) a source of at least one carbohydrate that provides rapidly available glucose;
(ii) a source of at least one carbohydrate that provides slowly available glucose; and
(iii) a source of at least one non-digestible carbohydrate or resistant starch.

17. The method of claim 16, wherein (i) the source of at least one carbohydrate that provides rapidly available glucose comprises at least one of:
(a) a monosaccharide;
(b) a glucose unit and a fructose unit joined by an α-1, β-2 glycosidic linkage;
(c) a glucose unit and a galactose unit joined by a β (1,4) glycosidic linkage;
(d) glucose units joined by α (1,4) glycosidic linkages;
(e) glucose units joined by α (1,6) glycosidic linkages; and
(f) oligosaccharides having a random mixture of α (1,2), α (1,3), α (1,4), and α (1,6) glycosidic linkages.

18. The method according to claim 17, wherein improving bone health is achieved by at least one of increasing bone density, increasing bone content, increasing bone length, improving bone microarchitecture, and combinations thereof.

19. The method according to claim 18, wherein improving bone microarchitecture includes at least one of increasing percent bone volume (BV/TV), increasing trabecular bone thickness (TbTh), increasing trabecular bone number (TbN), decreasing trabecular space (TbS), and combinations thereof.

20. The method of claim 19, wherein (i) the source of at least one carbohydrate that provides rapidly available glucose comprises at least one of glucose, fructose, tagatose, galactose, mannose, ribose, sucrose, maltose, isomaltose, lactose, isomalto-oligosaccharides, maltodextrin, and starch.

21. The method of claim 16, wherein (ii) the source of at least one carbohydrate that provides slowly available glucose comprises at least one of:
(i) a glucose unit and a fructose unit joined by an α (1,6) glycosidic linkage;
(ii) two glucose units joined by an α (1,1) glycosidic linkage;
(iii) a glucose unit and a fructose unit joined by an α (1,5) glycosidic linkage; and
(iv) oligosaccharides having alternating α (1,3) and α (1,6) glycosidic linkages.

22. The method of claim 21, wherein (ii) the source of at least one carbohydrate that provides slowly available glucose comprises at least one of isomaltulose, trehalose, sucromalt, and leucrose.

23. The method of claim 16, wherein (iii) the source of at least one non-digestible carbohydrate or resistant starch comprises at least one of:
(i) oligosaccharides having a random mixture of α (1,2), α (1,3), α (1,4), and β glycosidic linkages;
(ii) saccharides having linear chains of 2 to 60 fructose units or fructose polymers joined by β (2,1) glycosidic linkages; and
(iii) oligosaccharides having a random mixture of α (1,2), α (1,3), α (1,4), and α (1,6) glycosidic linkages.

24. The method of claim 23, wherein the source of at least one non-digestible carbohydrate or resistant starch comprises at least one of resistant maltodextrin, fructooligosaccharides, inulin, and isomalto-oligosaccharides.

25. The method of claim 16, wherein (i) the source of at least one carbohydrate that provides rapidly available glucose provides from 4% to 71% of the total calories supplied by carbohydrates in the nutritional composition, (ii) the source of at least one carbohydrate that provides slowly available glucose provides from 25% to 86% of the total calories supplied by carbohydrates in the nutritional composition, and (iii) the source of at least one non-digestible carbohydrate or resistant starch provides from 3% to 20% of the total calories supplied by carbohydrates in the nutritional composition.

26. The method of claim 16, wherein the nutritional composition further comprises at least one of a protein and a fat.

27. The method of claim 16, wherein the nutritional composition has a caloric density of 1 kcal/mL to 2 kcal/mL.

28. The method of claim 16, wherein the moderately malnourished individual suffers from poor bone health that includes at least one of decreased bone density, decreased bone content, decreased bone length, decreased bone microarchitecture, or combinations thereof.

29. The method according to claim 28, wherein the moderately malnourished individual is a preterm infant, infant, toddler, or child who suffers from decreased bone health, and the decreased bone health is caused by at least one of malnutrition, undernutrition, poor gut health, infectious disease, and compromised immune function.

30.-45. (canceled)
